(19) United States
(12) Patent Application Publication
(10) Pub. No.: US 2008/0051441 A1
(43) Pub. Date: Feb. 28, 2008

(54) Aryl Sulphonamide Modulators

(75) Inventors: Dean Brown, Wilmington, DE (US); Frances M McLaren, Wilmington, DE (US); Thomas Richard Simpson, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/722,752

(22) PCT Filed: Dec. 23, 2005
(86) PCT No.: PCT/SE05/02037

§ 371 Date: Jun. 25, 2007

Related U.S. Application Data
(60) Provisional application No. 60/639,514, filed on Dec. 28, 2004.

(51) Int. Cl. A61K 31/4245; A61K 31/42; A61P 25/00; A61P 25/16; A61P 25/18; A61P 25/22; A61P 25/24; A61P 25/28; A61P 25/34; A61P 43/00; C07D 261/04; C07D 271/06

(52) U.S. Cl. 514/364; 514/378; 548/131; 548/247

(57) ABSTRACT

Compounds of Formula I: 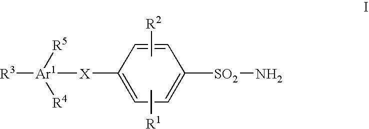
wherein R¹, R², R³, R⁴, R⁵ Ar¹ and X are as described in the specification, pharmaceutically-acceptable salts thereof, processes for preparing them, pharmaceutical compositions containing them and their use in therapy, especially for treatment of conditions associated with reductions in nicotinic transmission.

### TECHNICAL FIELD

The present invention relates to compounds or pharmaceutically-acceptable salts thereof, processes for preparing them, pharmaceutical compositions containing them and their use in therapy. The invention particularly relates to positive modulators of nicotinic acetylcholine receptors, such positive modulator having the capability to increase the efficacy of nicotinic receptor agonists.

### BACKGROUND OF THE INVENTION

Cholinergic receptors normally bind the endogenous neurotransmitter acetylcholine (ACh), thereby triggering the opening of ion channels. ACh receptors in the mammalian central nervous system can be divided into muscarinic (mAChR) and nicotinic (nAChR) subtypes based on the agonist activities of muscarine and nicotine, respectively. The nicotinic acetylcholine receptors are ligand-gated ion-channels containing five subunits. Members of the nAChR subunit gene family have been divided into two groups based on their amino acid sequences; one group containing so-called β subunits, and a second group containing α subunits. Three kinds of α subunits, α7, α8 and α9, have been shown to form functional receptors when expressed alone and thus are presumed to form homooligomeric pentameric receptors.

An allosteric transition state model of the nAChR has been developed that involves at least a resting state, an activated state and a “desensitized” closed channel state, a process by which receptors become insensitive to the agonist. Different nAChR ligands can stabilize the conformational state of a receptor to which they preferentially bind. For example, the agonists ACh and (−)-nicotine respectively stabilize the active and desensitized states.

Changes of the activity of nicotinic receptors has been implicated in a number of diseases. Some of these, for example myasthenia gravis and ADNFLE (autosomal dominant nocturnal front lobe epilepsy) are associated with reductions in the activity of nicotinic transmission either because of a decrease in receptor number or increased desensitization. Reductions in nicotinic receptors have also been hypothesized to mediate cognitive deficits seen in diseases such as Alzheimer's disease and schizophrenia.

The effects of nicotine from tobacco are also mediated by nicotinic receptors, and since the effect of nicotine is to stabilize receptors in a desensitized state, an increased activity of nicotinic receptors may reduce the desire to smoke.

Compounds which bind nACHrs have been suggested for the treatment of a range of disorders involving reduced cholinergic function such as Alzheimer's disease, cognitive or attention disorders, attention deficit hyperactivity disorders, anxiety, depression, smoking cessation, neuroprotection, schizophrenia, analgesia, Tourette's syndrome, and Parkinson's disease.

However, treatment with nicotinic receptor agonists which act at the same site as ACh is problematic because ACh not only activates, but also blocks receptor activity through processes which include desensitization and uncompetitive blockade. Furthermore, prolonged activation appears to induce a long-lasting inactivation. Therefore, agonists of ACh can be expected to reduce activity as well as enhance it.

At nicotinic receptors in general, and of particular note at the α7-nicotinic receptor, desensitization limits the duration of action of an applied agonist.

### DESCRIPTION OF THE INVENTION

We have found that certain compounds can increase the efficacy of agonists at nicotinic acetylcholine receptors (nAChR). Compounds having this type of action are those of formula I: 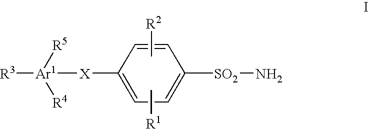
wherein:

Ar¹ is selected from aryl or heteroaryl where aryl is selected from phenyl or naphthyl and heteroaryl is selected from furyl, thienyl, imidazolyl, oxazolyl, thiazolyl, pyrrolyl, pyridyl, pyrazinyl, pyrimidinyl or quinolinyl;

R³, R⁴ and R⁵ are at each occurrence independently selected from hydrogen, C₁₋₄alkyl and C₁₋₄alkoxy;

X is selected from moieties according to formula II, III, IV, V, VI or VII

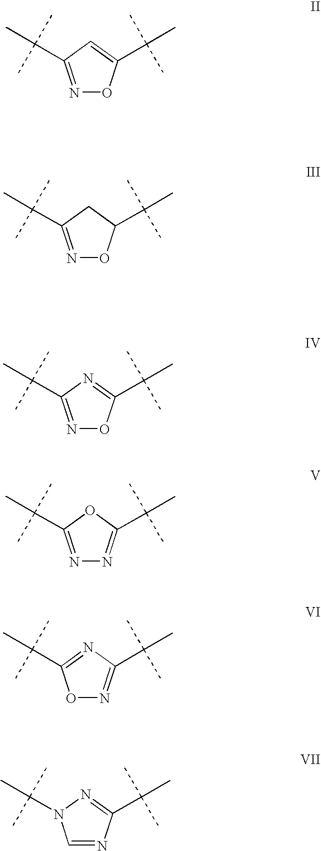

R¹ and R² are independently selected at each occurrence from hydrogen, halogen, —C₁₋₆alkyl, —C₁₋₆alkoxy, —C₂₋₆alkenyl, —C₂₋₆alkynyl, C₃₋₈cycloalkyl, —CN, —NO₂, —CF₃, —CONR³R⁴, —S(O)ₙR³ where n is o, 1 or 2, —NR³R⁴, —CH₂NR³R⁴, —OR³, —CH₂OR³ or —CO₂R³, where R³ and R⁴ at each occurrence are independently selected from hydrogen or C₁₋₄alkyl.

The invention also encompasses stereoisomers, enantiomers, in vivo-hydrolysable precursors and pharmaceutically-acceptable salts of compounds of formula I, pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes.

Compounds of the invention are positive modulators likely to be particularly useful for treatment of conditions associated with reductions in nicotinic transmission. In a therapeutic setting such compounds could restore normal interneuronal communication without affecting the temporal profile of activation. In addition, positive modulators are not expected to produce long-term inactivation of receptors as may the prolonged application of agonists.

In one aspect the invention encompasses compounds of formula I: 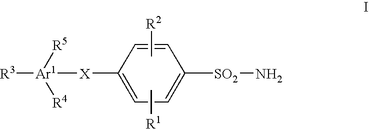
wherein:

Ar¹ is selected from aryl or heteroaryl where aryl is selected from phenyl or naphthyl and heteroaryl is selected from furyl, thienyl, imidazolyl, oxazolyl, thiazolyl, pyrrolyl, pyridyl, pyrazinyl, pyrimidinyl or quinolinyl;

R³, R⁴ and R⁵ are at each occurrence independently selected from hydrogen, C₁₋₄alkyl and C₁₋₄alkoxy;

X is selected from moieties according to formula II, III, IV, V, VI or VII

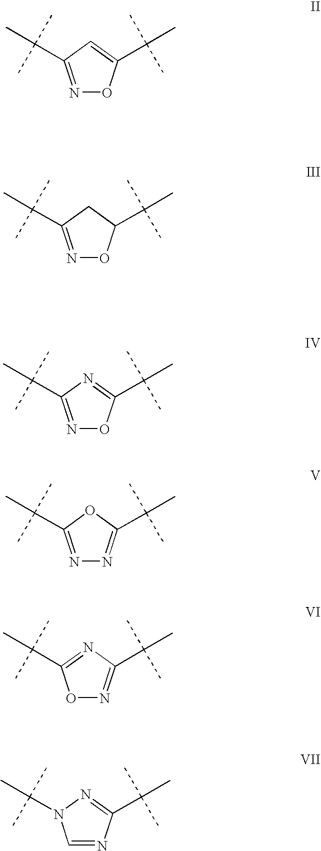

R¹ and R² are independently selected at each occurrence from hydrogen, halogen, —C₁₋₆alkyl, —C₁₋₆alkoxy, —C₂₋₆alkenyl, —C₂₋₆alkynyl, C₃₋₈cycloalkyl, —CN, —NO₂, —CF₃, —CONR³R⁴, —S(O)ₙR³ where n is o, 1 or 2, —NR³R⁴, —CH₂NR³R⁴, —OR³, —CH₂OR³ or —CO₂R³, where R³ and R⁴ at each occurrence are independently selected from hydrogen or C₁₋₄alkyl, and stereoisomers, enantiomers, in vivo-hydrolysable precursors and pharmaceutically-acceptable salts thereof.

A particular embodiment of this aspect of the invention includes compounds of formula I wherein:

Ar¹ is phenyl or pyridyl;

R³, R⁴ and R⁵ are at each occurrence independently selected from hydrogen, C₁₋₄alkyl and C₁₋₄alkoxy;

X is selected from moieties according to formula II, III, IV, V, VI or VII

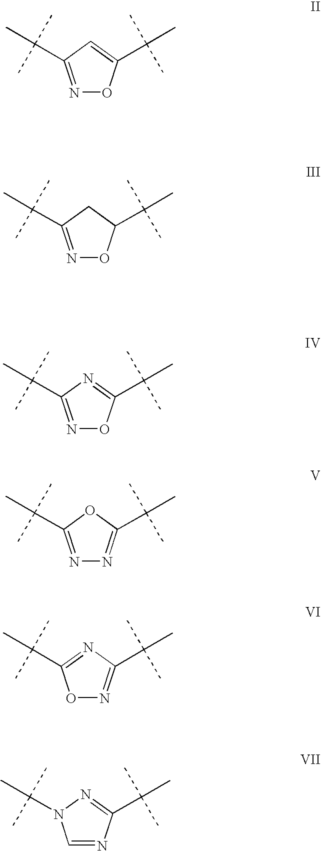

R¹ and R² are independently selected at each occurrence from hydrogen or halogen, and stereoisomers, enantiomers, in vivo-hydrolysable precursors and pharmaceutically-acceptable salts thereof.

Most particular compounds of the invention are those described herein.

In another aspect the invention is a method of treatment or prophylaxis of psychotic disorders, intellectual impairment disorders or diseases or conditions in which modulation of the α7 nicotinic receptor is beneficial, which method comprises administering a therapeutically-effective amount of a positive modulator of Formula I as described above or a diastereoisomer, enantiomer or pharmaceutically-acceptable salt thereof.

A particular aspect of the method of the invention is a method of treatment for Alzheimer's disease, learning deficit, cognition deficit, attention deficit, memory loss, Lewy Body Dementia, Attention Deficit Hyperactivity Disorder, anxiety, schizophrenia, mania, manic depression, Parkinson's disease, Huntington's disease, Tourette's syndrome, a neurodegenerative disorder in which there is loss of cholinergic synapse, jetlag, nicotine addiction, pain, ulcerative colitis or irritable bowel syndrome.

Methods of treatment of this invention include administering either a positive modulator as the only active substance, thus modulating the activity of endogenous nicotinic receptor agonists such as acetylcholine or choline, or administering a positive modulator together with a nicotinic receptor agonist.

In a particular form of this aspect of the invention, the method of treatment comprises treatment with an α7-nicotinic receptor modulator as described herein and an α7-nicotinic receptor agonist. An example of a suitable α7-nicotinic receptor agonist is (−)-spiro[1-azabicyclo[2.2.2.]octane-3,5′-oxazolidine]-2′-one. Other α7-nicotinic receptor agonists useful for treatment in conjunction with positive modulators of the present invention are described in international publications WO 96/06098, WO 97/30998 and WO 99/03859.

Another aspect of the invention comprises methods of preparing compounds according to Formula I.

Positive modulators of the invention have the advantage that they are less toxic, more efficacious, longer acting, have a broader range of activity, be more potent, produce fewer side effects, are more easily absorbed or have other useful pharmacological properties.

Acid addition salts re also within the scope of the invention. Such salts include salts of mineral acids, for example the hydrochloride and hydrobromide salts; and salts formed with organic acids such as formate, acetate, maleate, benzoate, tartrate, and fumarate salts. Acid addition salts of compounds of Formula I may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

The compounds of Formula I may exist in tautomeric or enantiomeric forms, all of which are included within the scope of the invention. The various optical isomers may be isolated by separation of a racemic mixture of the compounds using conventional techniques, for example by fractional crystallization, or chiral HPLC. Alternatively the individual enantiomers may be made by reaction of the appropriate optically active starting materials under reaction conditions which will not cause racemization.

A further aspect of the invention comprises a pharmaceutical composition for treating or preventing a condition or disorder as described herein arising from dysfunction of nicotinic acetylcholine receptor neurotransmission in a mammal, preferably a human. Such a pharmaceutical composition comprises a therapeutically-effective amount of a compound of Formula I, an enantiomer thereof or a pharmaceutically-acceptable salt thereof, effective in treating or preventing such disorder or condition and a pharmaceutically-acceptable carrier.

Another aspect of the invention is a pharmaceutical composition comprising a compound according to Formula I as described herein or a diastereoisomer, enantiomer or pharmaceutically-acceptable salt thereof, together with at least one pharmaceutically-acceptable diluent or carrier.

In particular, this aspect of the invention provides a pharmaceutical composition including preferably less than 80% and more preferably less than 50% by weight of a compound of the invention in admixture with a pharmaceutically-acceptable diluent or carrier.

Examples of diluents and carriers are:

for tablets and dragees: lactose, starch, talc, stearic acid;

for capsules: tartaric acid or lactose;

for injectable solutions: water, alcohols, glycerin, vegetable oils;

for suppositories: natural or hardened oils or waxes.

Yet another pharmaceutical composition of the invention comprises in addition a nicotinic receptor agonist.

Another aspect of the invention provides a process for the preparation of a pharmaceutical composition, which comprises incorporating the ingredients in a composition by conventional processes.

Yet a further aspect of the invention is the use of a compound according to Formula I, an enantiomer thereof or a pharmaceutically-acceptable salt thereof, for the preparation of a medicament.

A particular aspect of the invention is the use of a compound according to Formula I as described herein or a diastereoisomer, enantiomer or pharmaceutically-acceptable salt thereof, in the manufacture of a medicament for the treatment or prophylaxis of psychotic disorders, intellectual impairment disorders, human diseases or conditions in which modulation of the α7 nicotinic receptor is beneficial including Alzheimer's disease, learning deficit, cognition deficit, attention deficit, memory loss, Lewy Body Dementia, Attention Deficit Hyperactivity Disorder, anxiety, schizophrenia, mania, manic depression, Parkinson's disease, Huntington's disease, Tourette's syndrome, a neurodegenerative disorder in which there is loss of cholinergic synapse, jetlag, nicotine addiction, pain, ulcerative colitis or irritable bowel syndrome.

In a particular form, this aspect of the invention is the use of compound according to the invention in the manufacture of a medicament for the treatment or prophylaxis of a condition associated with reduced nicotinic receptor transmission or a condition associated with reduced nicotinic receptor density which could be one of the diseases or conditions mentioned herein, which treatment comprises administering said medicament comprising a therapeutically effective amount of a compound according to the invention to a patient.

It will be understood that this use includes the manufacture of medicaments comprising either a positive modulator as the only active substance providing modulation of the activity of endogenous nicotinic receptor agonists, or the manufacture of medicaments comprising a positive modulator in combination with a nicotinic receptor agonist. Thus, this use provides for the manufacture of medicaments containing a positive modulator and medicaments containing in addition a nicotinic receptor agonist.

In a particular form of this aspect of the invention, the medicament or pharmaceutical composition comprises an α7-nicotinic receptor modulator as described herein and an α7-nicotinic receptor agonist. An example of a suitable α7-nicotinic receptor agonist is (−)-spiro[1-azabicyclo[2.2.2.]octane-3,5′-oxazolidine]-2′-one. Other α7-nicotinic receptor agonists useful in medicaments in conjunction with positive modulators of the present invention are described in international publications WO 96/06098, WO 97/30998 and WO 99/03859.

Still a further aspect of the invention is a method of treating or preventing a condition or disorder in mammals and particularly humans as mentioned herein arising from dysfunction of nicotinic acetylcholine receptor neurotransmission.

A particular form of this aspect of the invention provides a method for the treatment of a condition associated with reduced nicotine transmission, by administering to a patient in need of such treatment, a medically effective amount of a positive modulator of a nicotinic receptor agonist, said positive modulator having the capability to increase the efficacy of the said nicotinic receptor agonist.

In the above-mentioned compositions, uses and methods, the amount of a compound according to Formula I employed will, of course, vary with the compound employed, the mode of administration and the treatment desired. However, in general, satisfactory results will be obtained when a compound of the invention is administered to provide a daily dosage of from about 0.1 mg to about 20 mg per kg of animal body weight, which may be given as divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carrier or diluent.

In compositions, uses and methods of the invention, a compound of Formula I, an enantiomer thereof, or a pharmaceutically-acceptable salts thereof, may be used on its own in the form of appropriate medicinal preparations for enteral or parenteral administration or may be used in a composition containing other pharmacologically-active agents. For example, a composition containing other pharmacologically-active agents may contain a positive modulator compound according to Formula I together with a nicotinic receptor agonist.

Accordingly, the invention includes compositions comprising a positive modulator as the only active substance, thus modulating the activity of endogenous nicotinic receptor agonists such as acetylcholine or choline, and compositions comprising a positive modulator in combination with a nicotinic receptor agonist. Thus, the said pharmaceutical compositions containing a positive modulator of a nicotinic receptor agonist may, in addition, comprise a nicotinic receptor agonist.

Examples of diseases or conditions for which aspects of the present invention are useful include schizophrenia, mania and manic depression, anxiety, Alzheimer's disease, learning deficit, cognition deficit, attention deficit, memory loss, Lewy Body Dementia, Attention Deficit Hyperactivity Disorder, Parkinson's disease, Huntington's disease, Tourette's syndrome, jetlag, and nicotine addiction (including that resulting from exposure to products containing nicotine).

It will be understood that the a positive modulator of the invention can be administered either with the purpose of modulating the action of endogenous nicotine receptor agonists such as acetylcholine or choline, or to modulate the action of an exogenous nicotinic receptor agonist.

Experimental Methods

The activity of the compounds of the invention may be measured in the tests set out below:

(a) Xenopus Oocyte Current Recording

Xenopus oocytes provided a powerful means of assessing the function of proteins thought to be subunits of ligand-gated ion-channels. Injection of RNA transcribed from cDNA clones encoding the appropriate receptor subunits, or injection of cDNA in which the coding sequence is placed downstream of a promoter, results in the appearance of functional ligand-gated ion-channels on the surface of the oocyte (see e.g. Boulter et al. (1987) Proc. Natl. Acad. Sci. U.S.A. 84, 7763-7767).

Consequently, one convenient technique to assess the enhancement of nicotinic efficacy is two-electrode voltage-clamp recording from Xenopus oocytes that express α7-nicotinic receptors from cRNA.

Xenopus laevis frogs (Xenopus I, Kalamazoo, Mich.) may be anesthetized using 0.15% tricaine. Oocytes are removed to OR2 solution (82 mM NaCl, 2.5 mM KCl, 5 mM HEPES, 1.5 mM NaH₂PO₄, 1 mM MgCl₂, 0.1 mM EDTA; pH 7.4). The oocytes are defolliculated by incubation in 25 mL OR2 containing 0.2% collagenase 1A (Sigma) two times for 60 min on a platform vibrating at 1 Hz and may be stored in Leibovitz's L-15 medium (50 μg/ml gentomycin, 10 Units/ml penicillin, and 10 μg/ml streptomycin). Approximately 50 ng of cRNA is injected into each oocyte on the following day.

Oocytes are placed in an external recording solution consisting of 90 mM NaCl, 1 mM KCl, 1 mM MgCl₂, 1 mM BaCl₂, 5 mM HEPES at pH 7.4. Two-electrode voltage-clamp recording may be carried out using an Oocyte Clamp amplifier (for example an OC 725C; Warner Instrument, Hamden, Conn.). Oocytes are impaled with two electrodes of 1-2 MΩ tip resistance filled with 3M KCl. Recordings are begun when membrane potential becomes stable at potentials negative to −20 mV (resting membrane potentials are less negative when Ba⁺⁺ replaces Ca⁺⁺ in bathing solutions). Membrane potential is clamped at −80 mV. Oocytes are continuously perfused at 5 mL/min with a recording solution with or without acetylcholine.

Current amplitude is measured from baseline to peak. EC₅₀ values, maximal effect, and Hill slopes may be estimated by fitting the data to the logistic equation using, for example, GraphPad Prism (GraphPad Software, Inc., San Diego, Calif.).

Increases in agonist efficacy elicited by a positive modulator can be calculated in two ways:

(1) As a percent potentiation of current amplitude which is defined as 100(Im−Ic)/Ic where Im is current amplitude in the presence of modulator and Ic is current in the absence of modulator.

(2) As a percent potentiation of “area under curve” of an agonist trace, which is the integration of net current over time. Area under the curve is a common representation of the total ion flux through the channel.

(b) Ca⁺⁺ Flux Imaging

Imaging of Ca⁺⁺ flux through nAChR α7 receptors transiently expressed in a cell line is another means of assaying modulator activity.

Cells expressing α7 receptors (for example HEK-293 cells or cell cultured neurons) are grown to confluence in 96 well plates and loaded with fluo-3, a fluorescent calcium indicator. To screen for α7 modulatory activity, a 96 well plate is placed in a fluorescence imaging plate reader (FLIPR) and test compounds along with an α7 agonist are applied simultaneously to all wells. Receptor activation is measured by calcium influx into cells which is quantified by the increase in fluorescence intensity of each well, as recorded simultaneously by the FLIPR. A modulatory effect is shown by an increase in fluorescence over that induces by agonist alone. Similarly, to test for nAChR α7 agonist activity, test compounds along with an α7 modulator are applied simultaneously to all wells. Receptor activation is measured by calcium influx into cells which is quantified by the increase in fluorescence intensity of each well. An agonist effect is determined by the increase in fluorescence over that induced by a modulator alone.

Cell-cultured neurons may be prepared as follows. Eighteen day old Sprague-Dawley rat fetuses (E-18) are aseptically removed from a pregnant female, sacrificed, the frontal cortices of the brains removed, the meninges stripped, and the cleaned cortex placed into cold HBSS. If hippocampal tissue is desired, the hippocampus is dissected away from the cortex and then placed into cold HBSS. The tissues are mechanically dispersed, washed once in HBSS (200 g for 30 min in 4° C.) resuspended in a Sato's medium supplemented with glutamine, antibiotics, potassium chloride, insulin, transferrin, selenium, and 5% heat-inactivated fetal bovine serum (FBS; endotoxin free) and plated into each of a 24-well plate (coated with poly-L-lysine). The wells may contain glass cover slips which are also coated with PLL. The plates are incubated at 37° C. in a CO₂ incubator. After 24 hours the medium is removed, fresh medium added, and the cells allowed to grow for at least another 11 days, feeding when necessary.

Compounds of the invention cause a 2-fold increase (100% potentiation) of baseline current as measured baseline to peak at low concentration of acetylcholine (30 μM), indicating that they are expected to have useful therapeutic activity. Compounds of the invention also increase the flux of Ca⁺⁺ when applied in the Ca2+ flux-imaging assay. Any increase of Ca⁺⁺ flux, caused by a compound of the invention, compared to the Ca⁺⁺ flux caused by an agonist alone (as measured in Fluorescence Intensity Units) indicates that they are expected to have useful therapeutic activity.

Compounds of the invention have the advantage that they may be less toxic, be more efficacious, be longer acting, have a broader range of activity, be more potent, produce fewer side effects, are more easily absorbed or have other useful pharmacological properties.

General Experimental Procedures

The invention is illustrated by, but not limited to, examples described herein in which descriptions, where applicable and unless otherwise stated, the following terms, abbreviations and conditions are used:

Commercial reagents were used without further purification.

The following abbreviations are used herein: aq., aqueous; atm, atmospheric pressure; BOC, 1,1-dimethylethoxycarbonyl; DCM, dichloromethane; DMF, N,N-dimethylformamide; DMSO, dimethyl sulphoxide; EtOH, ethanol; Et2O, diethyl ether; EtOAc, ethyl acetate; h, hour(s); HPLC, high pressure liquid chromatography; HOBT, 1-hydroxybenzotriazole; MeOH, methanol; min, minutes; MS, mass spectrum; NMR, nuclear magnetic resonance; psi, pounds per square inch; RT, room temperature; sat., saturated; TEA, triethylamine; TFA, trifluoroacetic acid; THF, tetrahydrofuran.

Temperatures are given in degrees Celsius (° C.); unless otherwise stated, operations were carried out at room or ambient temperature (18-25° C.).

Organic solutions were dried over anhydrous sodium or magnesium sulphate; evaporation of solvent was carried out using a rotary evaporator under reduced pressure (4.5-30 mm Hg) with a bath temperature of up to 60° C.

Chromatography means flash column chromatography on silica gel unless otherwise noted; solvent mixture compositions are given as volume percentages or volume ratios.

When given, NMR data is in the form of delta values for major diagnostic protons (given in parts per million (ppm) relative to tetramethylsilane as an internal standard) determined at 300 MHz.

Melting points are uncorrected.

Mass spectra were recorded using either a Hewlett Packard 5988A or a MicroMass Quattro-1 Mass Spectrometer and are reported as m/z for the parent molecular ion. Room temperature refers to 20-25° C.

Reactions described herein, unless otherwise noted, are usually conducted at a pressure of about one to about three atmospheres, preferably at ambient pressure (about one atmosphere).

Unless otherwise stated, the reactions are conducted under an inert atmosphere, preferably under a nitrogen atmosphere.

The compounds of the invention and intermediates may be isolated from their reaction mixtures by standard techniques.

As used herein, unless otherwise indicated, “C₁₋₆alkyl” includes methyl, ethyl, n-propyl, n-butyl, i-propyl, i-butyl, t-butyl, s-butyl, and the like, and C₃₋₈alkyl moieties may be straight-chained, branched or cyclic, for example cyclopropyl or cyclobutyl.

As used herein, unless otherwise indicated, “C₂₋₄alkenyl” includes but is not limited to 1-propenyl, 2-propenyl, 1-butenyl, 2-butenyl and 3-butenyl.

As used herein, unless otherwise indicated, “C₂₋₄alkynyl” includes but is not limited to ethynyl, 1-propynyl, 2-propynyl, 1-butynyl, 2-butynyl and 3-butynyl.

As used herein “halogen” means fluoride, chloride, bromide, or iodide.

### EXAMPLES

Compounds of the invention may be made generally by the process described in Examples 1 through 5. In all processes described herein, where necessary, hydroxy, amino or other reactive groups may be protected using a protecting group as will be understood by those of skill in the art.

### Example 1

### 4-[3-(2,4,6-Trimethyl-phenyl)-4,5-dihydro-isoxazol-5-yl]-benzenesulphonamide

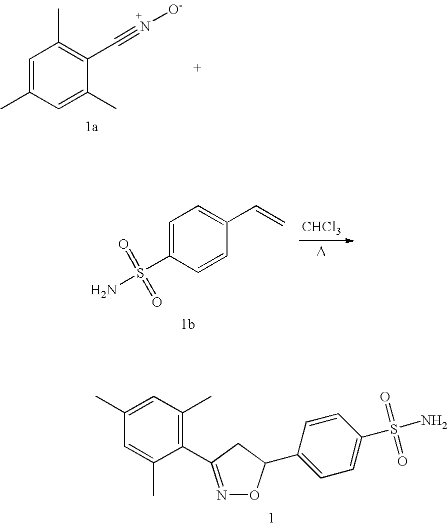

A stirred solution of mesitonitrile-N-oxide 1a (0.200 g, 1.23 mmol) and 4-vinyl-benzenesulphonamide 1b in chloroform was heated to reflux for 18 h. The residue was concentrated and purified using chromatography (SiO₂, CHCl₃/MeOH, gradient 0 to 10% in MeOH) to give the title compound as a solid (0.030, 7%): ¹H NMR (300 MHz, CDCl₃, 300K) δ 2.18 (s, 6H), 2.33 (s, 3H), 3.03-3.11 (br m, 1H), 3.63-3.72 (br m, 1H), 4.86 (br s, 2H), 5.80-5.85 (br ms, 1H), 6.88 (s, 2H), 7.57 (d, 2H, J=7.2 Hz), 7.95 (d, 2H, J=7.2 Hz. APCI (m+1), m/z=345. LC/MS=2.38.

### Example 2

### 2-Chloro-4-[3-(2,4,6-trimethyl-phenyl)-isoxazol-5-yl]-benzenesulphonamide

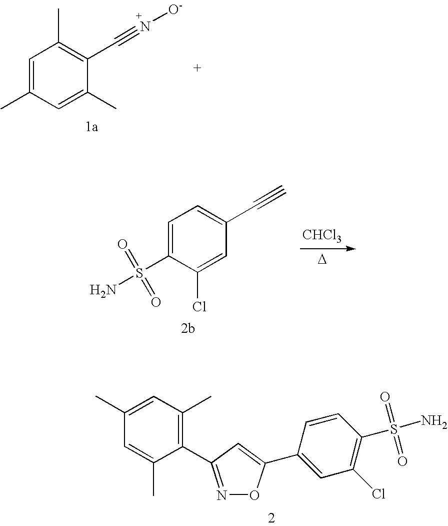

A stirred solution of mesitonitrile-N-oxide 1a (0.073 g, 0.33 mmol) and 2-chloro-4-ethynyl-benzenesulphonamide 2b (0.034 g, 0.33 mmol) in chloroform was heated to reflux for 18 h. The residue was concentrated and purified using chromatography (SiO₂, EtOAc/Hex, gradient 0 to 20% in EtOAc) to give the title compound as a solid (0.037, 28%): ¹H NMR (300 MHz, CDCl₃, 300K) δ 2.18 (s, 6H), 2.33 (s, 3H), 5.18 (br s, 2H), 6.62 (s, 1H), 6.97 (s, 2H), 7.85 (d, 1H, J=8.0 Hz), 8.02 (s, 1H), 8.23 (d, 1H, J=8.0 Hz). APCI (m+1), m/z=377. LC/MS=2.71.

### Preparation of 2-chloro-4-ethynyl-benzenesulphonamide 2b

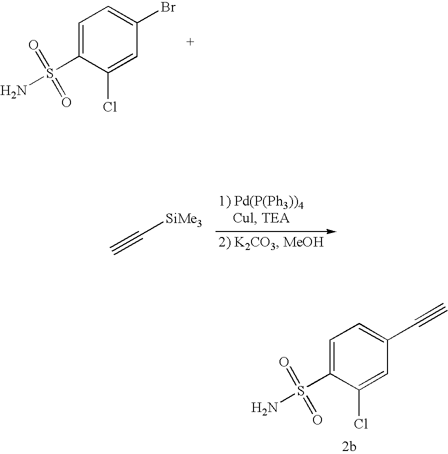

To a stirred solution of 2-chloro-4-bromo-benzenesulphonamide 2d (1.02 g, 3.77 mmol) and trimethlysilylacetylene (0.55 g, 5.66 mmol) in triethylamine (24 mL) was added copper (I) iodide (0.028 g, 0.14 mmol) and tetrakistriphenylphosphino palladium (0) (0.063 g, 0.05 mmol). The mixture was heated to 80° C. for 18 h. The mixture was concentrated and diethyl ether was added to the residue. The solid obtained was filtered off. To the solid was added MeOH (100 mL) and K₂CO₃ (8.66 mmol) and the reaction was stirred for 18 h. The reaction was concentrated, and partitioned between CHCl₃ and H₂O. A tan solid precipitated, was recovered by filtration and used without further purification.

### Example 3

### 3-Fluoro-4-[3-(2,4,6-trimethyl-phenyl)-isoxazol-5-yl]-benzenesulphonamide

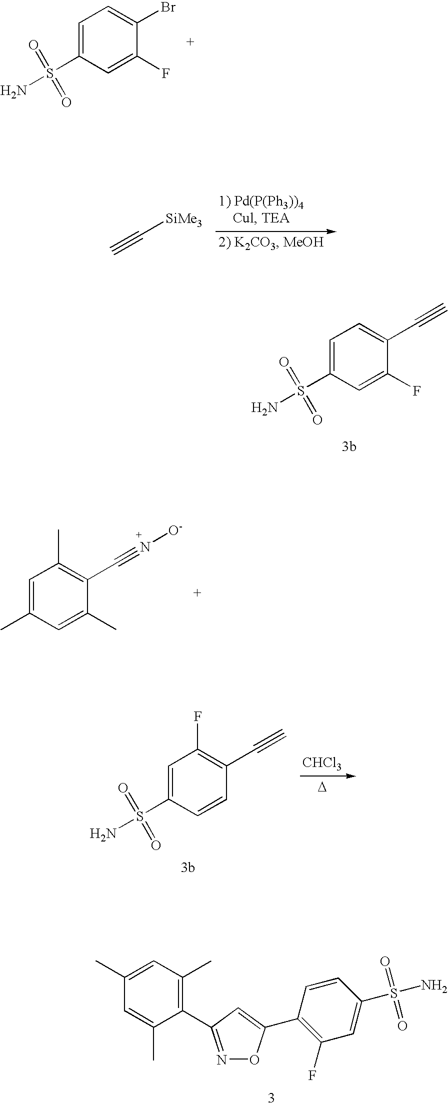

Example 3 was prepared in analogous fashion to Example 2 using 3-chloro-4-ethynyl-benzenesulphonamide 3b. The material was isolated with chromatography (CH₂Cl₂, SiO₂) and then purified again on preparatory HPLC (acetonitrile/H₂O gradient) to give the title compound as a white solid (0.012 g, 6%): ¹H NMR (300 MHz, CDCl₃, 300K) δ 2.18 (s, 6H), 2.33 (s, 3H), 5.00 (br s, 2H), 6.78 (d, J=4.2 Hz), 1H), 6.96 (s, 1H), 7.77 (d, 1H, J=11.0 Hz), 7.85 (s, J=8.4 Hz, 1H), 8.19-8.24 (m, 1H). APCI (m+1), m/z=361. LC/MS=2.42.

### Example 4

### 4-[3-(2,4,6-trimethyl-phenyl)-isoxazol-5-yl]-benzenesulphonamide

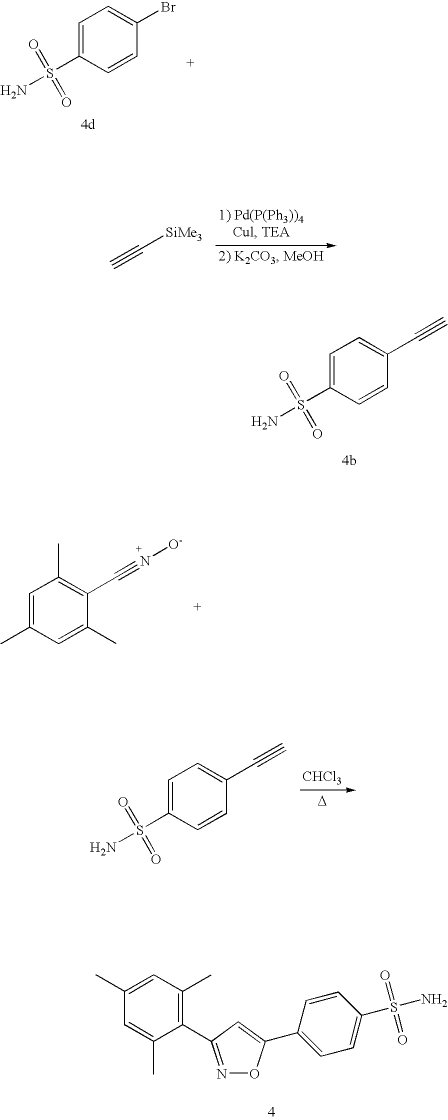

Example 4 was prepared in analogous fashion to Example 2 using 4-ethynyl-benzenesulphonamide 4b. The material was isolated with chromatography (EtOAc/Hexane 20/80, SiO₂) to give the title compound as a white solid (0.011 g, 8%): ¹H NMR (300 MHz, CDCl₃, 300K) δ 2.18 (s, 6H), 2.33 (s, 3H), 4.86 (br s, 2H), 6.59 (s, 1H), 6.96 (s, 2H), 7.95 (d, J=8.4 Hz, 2H), 8.06 (d, J=8.4 Hz, 2H). APCI (m+1), m/z=343. LC/MS=2.60.

### Example 5

### 2-Fluoro-4-[3-(2,4,6-trimethyl-phenyl)-isoxazol-5-yl]-benzenesulphonamide

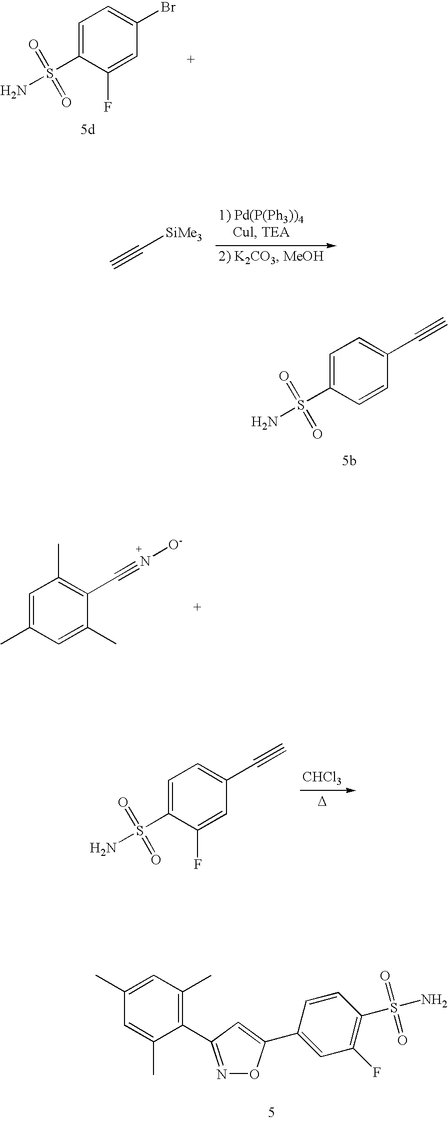

Example 5 was prepared in analogous fashion to Example 2 using 4-ethynyl-benzenesulphonamide 5b. The material was isolated with chromatography (EtOAc/Hexane 20/80, SiO₂) to give the title compound as a white solid (0.091 g, 30%): 1H NMR (300 MHz, CDCl₃, 300K) δ 2.18 (s, 6H), 2.33 (s, 3H), 5.10 (br s, 2H), 6.60 (s, 1H), 6.96 (s, 2H), 7.68-7.73 (br m, 2H), 8.03 (app t, J=7.5 Hz, 1H). APCI (m+1), m/z=361. LC/MS=2.65.

The compounds of examples 6 and 7 may be prepared using procedures substantially analogous to those described in the foregoing Examples.

### Example 6

### 4-[3-(2,4,6-Trimethyl-phenyl)-[1,2,4]oxadiazol-5-yl]-benzenesulphonamide

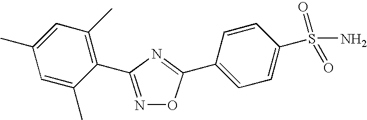

### Example 7

### 4-[3-(4-Methoxy-2,3-dimethyl-phenyl)-4,5-dihydro-isoxazol-5-yl]-benzenesulphonamide

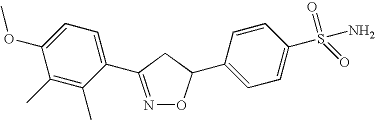

1. A compound of formula I: 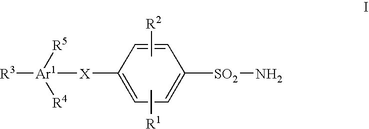
wherein:
Ar¹ is selected from aryl or heteroaryl where aryl is selected from phenyl or naphthyl and heteroaryl is selected from furyl, thienyl, imidazolyl, oxazolyl, thiazolyl, pyrrolyl, pyridyl, pyrazinyl, pyrimidinyl or quinolinyl;
R³, R⁴ and R⁵ are at each occurrence independently selected from hydrogen, C₁₋₄alkyl and C₁₋₄alkoxy;
X is selected from moieties according to formula II, I, IV, V, VI or VII 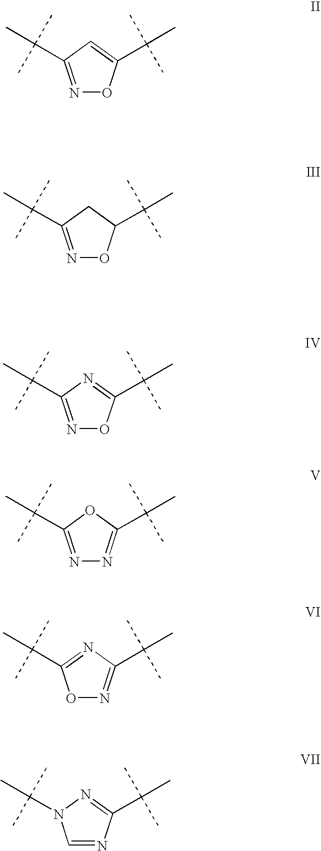
R¹ and R² are independently selected at each occurrence from hydrogen, halogen, —C₁₋₆alkyl, —C₁₋₆alkoxy, —C₂₋₆alkenyl, —C₂₋₆alkynyl, C₃₋₈cycloalkyl, —CN, —NO₂, —CF₃, —CONR³R⁴, —S(O)ₙR³ where n is o, 1 or 2, —NR³R⁴, —CH₂NR³R⁴, —OR³, —CH₂OR³ or —CO₂R³, where R³ and R⁴ at each occurrence are independently selected from hydrogen or C₁₋₄alkyl, and stereoisomers, enantiomers, in vivo-hydrolysable precursors and pharmaceutically-acceptable salts thereof.

2. A compound according to claim 1, wherein:
Ar¹ is phenyl or pyridyl;
R³, R⁴ and R⁵ are at each occurrence independently selected from hydrogen, C₁₋₄alkyl and C₁₋₄alkoxy;
X is selected from moieties according to formula II, III, IV, V, VI or VII 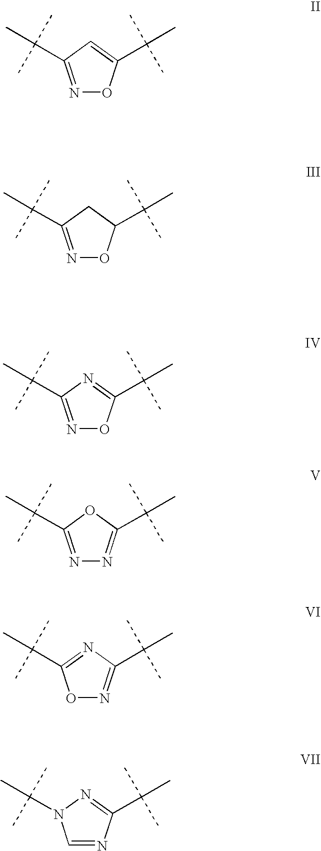
R¹ and R² are independently selected at each occurrence from hydrogen or halogen, and stereoisomers, enantiomers, in vivo-hydrolysable precursors and pharmaceutically-acceptable salts thereof.

3. A compound according to claim 1 selected from:
4-[3-(2,4,6-Trimethyl-phenyl)-4,5-dihydro-isoxazol-5-yl]-benzenesulfonamide;
2-Chloro-4-[3-(2,4,6-trimethyl-phenyl)-isoxazol-5-yl]-benzenesulfonamide;
3-Fluoro-4-[3-(2,4,6-trimethyl-phenyl)-isoxazol-5-yl]-benzenesulfonamide;
4-[3-(2,4,6-trimethyl-phenyl)-isoxazol-5-yl]-benzenesulfonamide;
2-Fluoro-4-[3-(2,4,6-trimethyl-phenyl)-isoxazol-5-yl]-benzenesulfonamide;
4-[3-(2,4,6-Trimethyl-phenyl)-[1,2,4]oxadiazol-5-yl]-benzenesulfonamide, and
4-[3-(4-Methoxy-2,3-dimethyl-phenyl)-4,5-dihydro-isoxazol-5-yl]-benzenesulfonamide.

4. A method of treatment or prophylaxis of a disease or condition in which modulation of the α7 nicotinic receptor is beneficial which method comprises administering a therapeutically-effective amount of a compound according to claim 1 to a subject suffering from said disease or condition.

5. The method of claim 4, wherein said disease or condition is anxiety, schizophrenia, mania or manic depression.

6. A method of treatment or prophylaxis of neurological disorders, psychotic disorders or intellectual impairment disorders, which comprises administering a therapeutically effective amount of a compound according to claim 1.

7. The method of claim 6, wherein said disorder is Alzheimer's disease, learning deficit, cognition deficit, attention deficit, memory loss, Attention Deficit Hyperactivity Disorder, Parkinson's disease, Huntington's disease, Tourette's syndrome, neurodegenerative disorders in which there is loss of cholinergic synapses, jetlag, nicotine addiction, craving, pain, or ulcerative colitis.

8. A method for inducing the cessation of smoking comprising administering an effective amount of a compound according to claim 1.

9. A pharmaceutical composition comprising a compound according to claim 1 and a pharmaceutically-acceptable diluent, lubricant or carrier.

10. A method of treatment or prophylaxis of a disease or condition in which activation of the α7 nicotinic receptor is beneficial which method comprises administering a therapeutically-effective amount of a pharmaceutical composition according to claim 9 to a subject suffering from said disease or condition.

11. The method of claim 10, wherein said disease or condition is anxiety, schizophrenia, mania or manic depression.

12. A method of treatment or prophylaxis of neurological disorders, psychotic disorders or intellectual impairment disorders, which comprises administering a therapeutically effective amount of a pharmaceutical composition according to claim 9.

13. The method of claim 12, wherein said disorder is Alzheimer's disease, learning deficit, cognition deficit, attention deficit, memory loss, Attention Deficit Hyperactivity Disorder, Parkinson's disease, Huntington's disease, Tourette's syndrome, neurodegenerative disorders in which there is loss of cholinergic synapses, jetlag, nicotine addiction, craving, pain, or ulcerative colitis.

14. A method for inducing the cessation of smoking comprising administering an effective amount of a pharmaceutical composition according to claim 9.

15-16. (canceled)
